SEQUENCE LISTING (2 sequences)

SEQ ID NO: 1 - DNA, 21 bp, Artificial Sequence
  HBV forward primer
atgcaactttttcacctctgc

SEQ ID NO: 2 - DNA, 21 bp, Artificial Sequence
  HBV reverse primer
ggagactctaaggcttcccga

(19) United States
(12) Patent Application Publication
(10) Pub. No.: US 2013/0078611 A1
(43) Pub. Date: Mar. 28, 2013

(54) METHOD FOR DETECTING MICROORGANISMS AND A KIT THEREOF

(71) Applicants: George Chin Sheng Chou, Tainan (TW); Yu-Liang Su, Tainan (TW)

(72) Inventors: George Chin Sheng Chou, Tainan (TW); Yu-Liang Su, Tainan (TW)

(21) Appl. No.: 13/245,592

(22) Filed: Sep. 26, 2011

(51) Int. Cl. C12Q 1/70; C12Q 1/68

(52) U.S. Cl. 435/5; 435/6.12

(57) ABSTRACT

The present invention provides a method for detecting microorganisms comprising steps of PCR, magbead, complex forming, blocking and washing and reporting, in which microorganisms, particular to HBV, can be easily detected via a time-saving and user-friendly process, with high sensitivity and stability. Furthermore, according to the method for detecting microorganisms, a kit for detecting microorganisms is also provided in the present invention, so that the detection of HBV can be achieved conveniently and easily.

### BACKGROUND OF THE INVENION

1. Field of the Invention

The present invention relates to a method for detecting microorganisms and a kit thereof, particularly to a method for detecting hepatitis B virus (HBV) and a kit thereof.

2. Description of the Related Art

Human hepatitis B virus (HBV) is considered to be one of the most significant viral pathogen worldwide, especially in Asia, and which leads to severely liver diseases, such as acute hepatic inflammation, fulminant hepatitis and liver cirrhosis, and also increases the risk in the development of primary hepatocellular carcinoma.

In clinical medicine, the conventional diagnosis of HBV is mainly based on detecting a surface antigen thereof which is also known as HBsAg, and an antigen of core protein named HBeAg. However, the said antigens of the HBV comprise an antigenic determinant (AD), as a crucial part of antigens and being recognized by immune system, and which is easily mutated under various circumstances. Accordingly, the conventional diagnosis by detecting the antibodies specific to the said antigens may prone to false negative reaction data and diagnosis error, so that the incidence of serious complication of HBV infection, as well as the difficulties in the therapy and prophylaxis may both increase due to the disadvantage of conventional diagnosis.

On the other hand, polymerase chain reaction (PCR) is another useful strategy in detecting microorganisms, HBV for example, by amplifying and detecting specific fragments of particular microorganisms. In conventional arts, the specific fragments obtained from PCR are analyzed via either gel electrophoresis or fluorescence staining, which makes the detection of HBV being processed in a time-consuming and labor-intensive process.

Hence there is a need of providing a user-friendly diagnostic method and apparatus for detecting microorganisms, for the sake of improving the disadvantages of conventional art, and detecting various microorganisms with high efficiency and quality.

### SUMMARY OF THE INVENTION

The primary objective of this invention is to provide a method for detecting microorganisms, in which the detection of HBV can be processed with an easy platform, so as to be time-saving and user-friendly.

The secondary objective of this invention is to provide a kit for detecting microorganisms, so that the detection of HBV can be achieved conveniently and cost-effectively.

A method for detecting microorganisms comprises steps of: PCR, by amplifying a target DNA fragment of a microorganism via polymerase chain reaction, and obtaining PCR fragments with first affinity molecules labeled on two ends of the PCR fragments; magbead, by providing a plurality of magnetic beads, with the plurality of magnetic beads having second affinity molecules immobilized on surfaces of each of the magnetic beads and with the second affinity molecules having specific affinity to the first affinity molecules; complex forming, by co-incubating the PCR fragments with the plurality of magnetic beads, with the PCR fragments binding and interacting to the second affinity molecules on the magnetic beads through the first affinity molecules on one ends of the PCR fragments, with the first affinity molecules on the other ends of the PCR fragments being free from the magnetic beads and with the binding of the first and the second affinity molecules forming complexes of PCR fragment-magnetic bead; blocking and washing, by providing a blocking buffer and a washing buffer to remove un-bound PCR fragments or impurities, with the blocking buffer resuspending the complexes of PCR fragment-magnetic bead, and with the washing buffer washing the complexes of PCR fragment-magnetic bead; and reporting, by providing an enzyme explorer labeled with third affinity molecules, with the third affinity molecules specifically binding to the first affinity molecules on the other ends of the PCR fragments to form complexes of enzyme explorer-PCR fragment-magnetic bead, and with the complexes of enzyme explorer-PCR fragment-magnetic bead being identified through the enzyme explorer; wherein the third affinity molecules on the enzyme explorer are different from the second affinity molecules and have specific affinity to the first affinity molecules on the PCR fragments.

A kit for detecting microorganisms comprises a pair of primer, comprising specific sequence to a target microorganism; a plurality of magnetic beads, having immobilized streptavidin on surfaces thereof; and an enzyme explorer comprising avidin conjugated luminescences; wherein the pair of primer has biotin labeled on 5′ ends.

Further scope of the applicability of the present invention will become apparent from the detailed description given hereinafter. However, it should be understood that the detailed description and specific examples, while indicating preferable embodiments of the invention, are given by way of illustration only, since various more will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 is a flowchart illustrating the method for detecting microorganisms in the present invention;

FIG. 2 is a diagram illustrating the binding between streptavidin-magnetic beads and biotinylated PCR fragments of the present invention;

FIG. 3 is a bar chart illustrating the quality of analysis of the method for detecting microorganisms in the present invention;

FIG. 4 is a bar chart illustrating the stability of streptavidin-magnetic beads of the present invention.

All figures are drawn for ease of explaining the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions conforming to specific force, weight, strength, and similar requirements will likewise be within the skill of the art after the following teachings of the present invention have been read and understood.

### DETAILED DESCRIPTION OF THE INVENTION

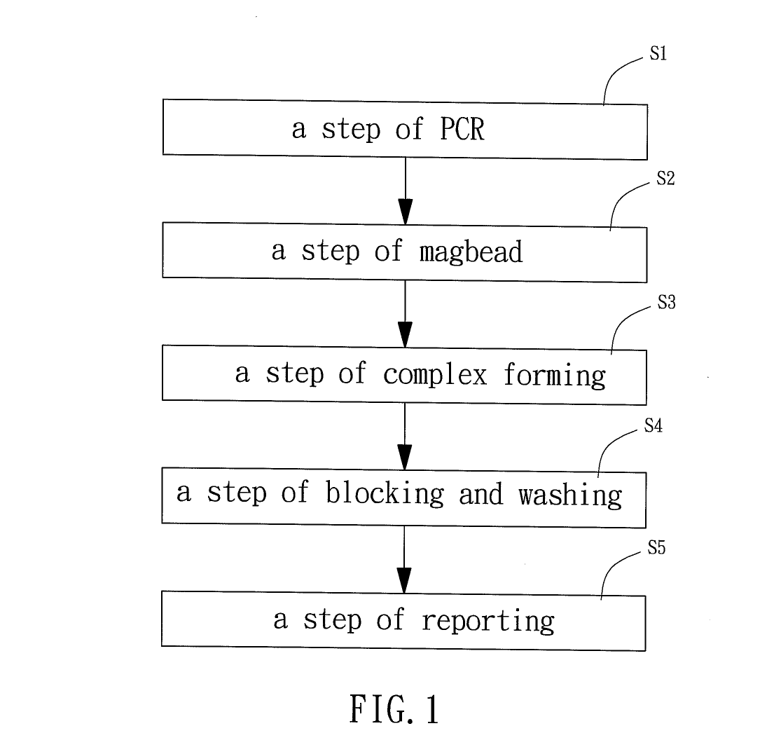

With reference to FIG. 1, the present invention relates to a method for detecting microorganisms comprises a step of “PCR S1,” a step of “magbead S2,” a step of “complex forming S3,” a step of “blocking and washing S4,” and a step of “reporting S5.” In the step of “PCR S1,” a target DNA fragment of a microorganism is amplified by polymerase chain reaction to obtain PCR fragments of the microorganism, wherein each end of the each PCR fragment have a first affinity molecule labeled on respectively. In specification, a pair of primer is designed according to a specific region of the microorganism and is used in PCR amplification of the step of “PCR S1.” The pair of primer comprises the first affinity molecules labeled on the 5′ ends of forward and reverse primer. With such arrangement, the PCR fragments having the first affinity molecules labeled on two ends thereof are obtained in the step of “PCR S1.” In the present invention, the microorganisms can be any possible pathogen, including virus, bacteria and fungi, preferably for hepatitis B virus (HBV).

In the step of “magbead S2,” a plurality of magnetic beads is prepared, with second affinity molecules immobilizing on surfaces of each of the plurality of magnetic beads. In specification, the second affinity molecules have specific affinity to the first affinity molecules, wherein the first and the second affinity molecules can be antigen and antibody, or biotin and avidin, preferably for biotin and streptabidin in the present invention.

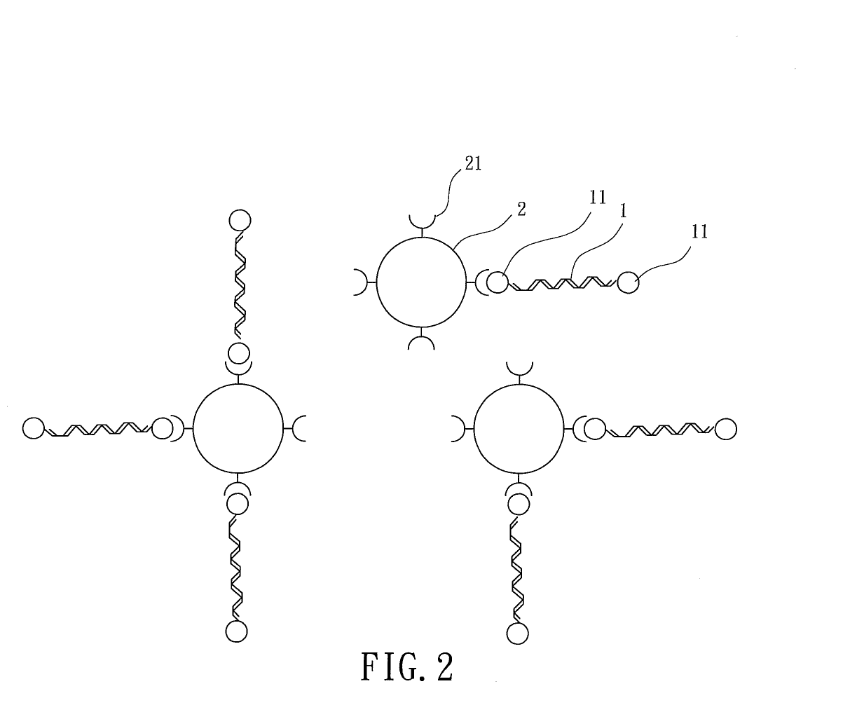

In the step of “complex forming S3,” the PCR fragments and the magnetic beads are co-incubated with each other, with the PCR fragments binding and immobilizing on the surfaces of the magnetic beads through the affinity between the first and the second affinity molecules and with the specific affinity of the first and the second affinity molecules forming complexes of PCR fragment-magnetic beads. In specification, the plurality of magnetic beads is resuspended in a binding buffer at first, followed by co-incubating with the PCR fragments at room temperature for 10 to 30 minutes. In this way, the PCR fragments are immobilized with the magnetic beads in accordance with the interaction between the first and the second affinity molecules as it is shown in FIG. 2. In the present invention, the binding buffer comprises 10 mM Tris-HCl (pH 7.5), 1 mM EDTA, and 2M NaCl.

Referring to FIG. 2, it is indicated that the bindings of the PCR fragments 1 and the plurality of magnetic beads 2 are established on the affinity between the first affinity molecules 11 labeled on the PCR fragments 1 and the second affinity molecule 21 immobilized on the plurality of magnetic beads. In the present invention, the first and the second affinity molecules 11, 21 are but not limit to streptavidin and biotin. Due to the fastest and strongest non-covalent biological interaction between the streptavidin and biotin, the biotinylated molecules, such as the PCR fragments 1 of the present invention, is sufficient to bind on surfaces of the streptavidin magnetic beads with only one end of each biotinylated molecule, and the other end of each biotinylated molecules is free from the magnetic beads and capable of being further response with any interacting target molecules.

In the step of “blocking and washing S4,” a blocking buffer and a washing buffer are prepared and used to remove un-bound PCR fragments or impurities. Specifically, the complexes of PCR fragment-magnetic bead are collected by using a magnetic separation rack and the supernatant are discarded. As following, the complexes of PCR fragment-magnetic bead are sequentially processed by repeatedly washing with the washing buffer, incubating in the blocking buffer at room temperature for 10 minutes, then recollecting the complexes of PCR fragment-magnetic bead with the magnetic separation rack and repeatedly washed the collected complexes of PCR fragment-magnetic bead with the washing buffer, and finally, resuspend the complexes of PCR fragment-magnetic bead in 1× PBS buffer. Moreover, the washing buffer of the present invention comprise 10 mM Tris-HCl (pH 7.5), 1 mM EDTA, and 2M NaCl, and the blocking buffer comprises 1× PBS buffer and substrates.

In the step of “reporting S4,” an enzyme explorer labeled with third affinity molecules is prepared and specifically interacted with the complexes of PCR fragment-magnetic beads via specific affinity between the first affinity molecules and the third affinity molecules, and accordingly enzyme explorer-PCR fragment-magnetic bead complexes are performed and identified by luminometer. In specification, the complexes of PCR fragment-magnetic beads are incubated in the enzyme explorer, with the third affinity molecules labeled on the enzyme explorer that is specific interacting with the free ends of first affinity molecules on the complexes of PCR fragment-magnetic beads, and therefore, the enzyme explorer-PCR fragment-magnetic bead complexes are performed and measured by luminometer. In the present invention, the third affinity molecules have specific affinity to the first affinity molecules, preferably being different from the second affinity molecules, and the third and the first affinity molecules are but not limit to avidin and biotin. Furthermore the enzyme explorer of the present invention can be any common fluorescent labels, radioactive, or chemiluminescent, preferably being avidin-conjugated horse reddish peroxidase (AV-HRPo) in the present invention. With such arrangement, AV-HRPo-biotinylated molecules-sterptavidin magnetic beads complexes are formed, and which are fast and easily analyzed via the luminometer or other measurement.

The method for detecting microorganisms in the present invention can be successfully applied in diagnosis of any body fluid, including blood, serum, and other samples for detecting any possible pathogen, HBV in particular.

In order to validate the efficiency of the method for detecting microorganisms of the present invention, a serial of trials is prepared and carried out by providing a sample of a microorganism, assigning the sample into to a plurality of groups, with each group containing various concentration of the sample individually, and determining and quantifying the microorganism of each group via the method of the present invention.

In the present embodiment, a constructed HBV positive control plasmid (pHBV-300,106) is prepared and assigned into 10 groups, which comprise various concentration of the pHBV-300,106, such as 4×, 8×, 16×, 32×, 64×, 128×, 256×, 512×, 1024×, and 2048×. The 10 groups of the pHBV-300,106 and a group of negative control are detected by using the method of the present invention. In the present embodiment, a pair of primer for detecting HBV, as set forth in SEQ ID NO. 1 and 2, is designed and used in the step of PCR of present embodiment, and which has biotin labeled on 5′ ends of forward and reverse primers.

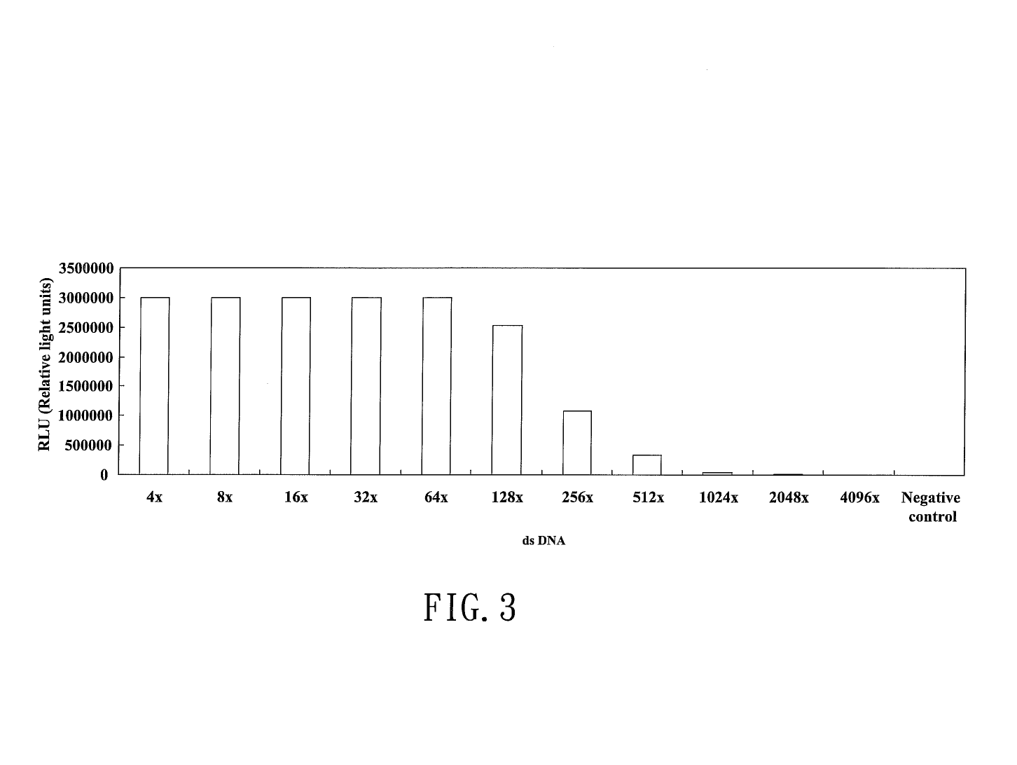

According to FIG. 3 and TABLE 1, it is noted that the method of the present invention is sufficient to detect HBV even at the lowest level of 0.3125 ng, so that the detection of HBV can be achieved easily and highly sensitively via the method of the present invention.

TABLE 1
Qualitative analysis of HBV dsDNA

| | 4× | 8× | 16× | 32× | 64× |
|---|---|---|---|---|---|
| | (40 ng) | (20 ng) | (10 ng) | (5 ng) | (2.5 ng) |
| No. folds | >569.6 | >569.6 | >569.6 | 502.8 | 460.3 |
| to NC | | | | | |
| | 128× | 256× | 512× | 1024× | 2048× |
| | (1.25 ng) | (0.625 ng) | (0.3125 ng) | (0.15625 ng) | (0.078125 ng) |
| No. folds | 222.8 | 91.3 | 7.4 | 1.6 | 0.3 |
| to NC | | | | | |

Additionally, the stability of the streptavidin magnetic beads is also demonstrated in the present embodiment by providing a batch of streptavidin magnetic beads, incubating at 4° C. and 25° C. for 7 days, 14 days and 30 days respectively, and then examining the pHBV-300,106 in various concentration (32×, 64×, 128× and 256×) via the method of the present invention.

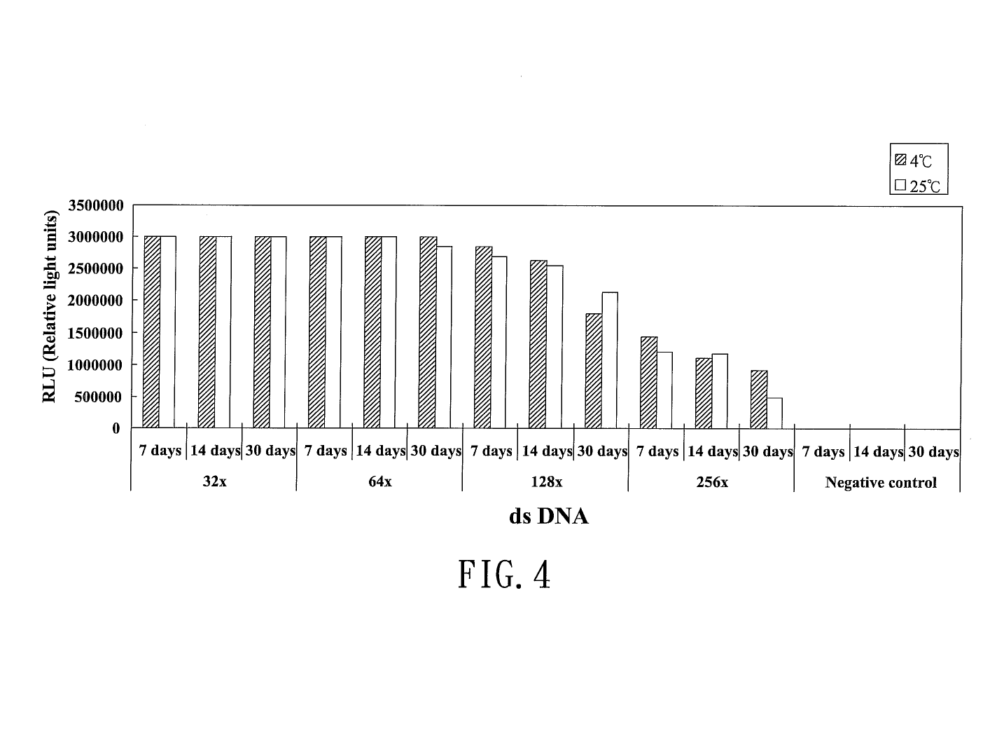

In FIG. 4, the detection data obtained from each batch and a negative control are summarized. It is suggested that there is no obvious difference between the data from 4° C. and 25° C.-incubating magnetic beads, and also the sensitivity of the streptavidin magnetic beads in detecting HBV is still maintained even after days of storage. Moreover, it is indicated that the streptavidin magnetic beads of the present invention are quite stable at room temperature, and accordingly there are allowable to be transport to anywhere under a general shipping condition.

Through the present invention, a method for detecting HBV is provided by carry out steps of PCR, magbead, complex forming, blocking and washing and reporting, in which microorganisms, particular to HBV, can be easily detected via a time-saving and user-friendly process with high sensitivity and stability. In this way, a kit for detecting microorganisms based on the method described above can be also provided in the present invention, and which comprises a pair of primer, comprising specific sequence to a target microorganism, such as HBV; a plurality of magnetic beads, having immobilized streptavidin on surfaces thereof; and an enzyme explorer comprising avidin conjugated luminescence for easily measuring. In the kit, the pair of primer has biotin labeled on 5′ ends, and is specific to the target microorganism. In the present invention the target microorganism can be virus, bacteria and fungi, preferably for hepatitis B virus (HBV). Furthermore, the plurality of magnetic bead of the kit have immobilized streptavidin on its surfaces, and which is capable of being bound and interacted with any biotinylated molecules. In this way, the biotinylated molecules are easily collected and analyzed for detecting microorganisms. Also, the enzyme explorer of the kit is but not limit to avidin conjugated luminescence, such as avidin conjugated horse reddish peroxidase (AV-HRPo). With the performance of the avidin conjugated luminescence of the kit, complexes of biotinylated molecules-streptavidin magnetic bead-avidin conjugated luminescence are fast formed and easy to analyze by a luminometer.

Also, the kit for detecting microorganisms of the present invention can further comprises a washing buffer, a blocking buffer and a binding buffer, wherein the said buffers are all easily purchased from any commercial supplies, Biolab for example, or easily prepared by users. In the present invention, the binding buffer and the washing buffer of the kit comprise 10 mM Tris-HCl (pH 7.5), 1 mM EDTA, and 2M NaCl, and the blocking buffer comprise 1× PBS buffer and substrates.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. A method for detecting microorganisms comprising steps of:
PCR, by amplifying a target DNA fragment of a microorganism via polymerase chain reaction, and obtaining PCR fragments with first affinity molecules labeled on two ends of the PCR fragments;
magbead, by providing a plurality of magnetic beads, with the plurality of magnetic beads having second affinity molecules immobilized on surfaces of each of the magnetic beads and with the second affinity molecules having specific affinity to the first affinity molecules;
complex forming, by co-incubating the PCR fragments with the plurality of magnetic beads, with the PCR fragments binding and interacting to the second affinity molecules on the magnetic beads through the first affinity molecules on one ends of the PCR fragments, with the first affinity molecules on the other ends of the PCR fragments being free from the magnetic beads and with the binding of the first and the second affinity molecules forming complexes of PCR fragment-magnetic bead;
blocking and washing, by providing a blocking buffer and a washing buffer to remove un-bound PCR fragments or impurities, with the blocking buffer resuspending the complexes of PCR fragment-magnetic bead, and with the washing buffer washing the complexes of PCR fragment-magnetic bead; and
reporting, by providing an enzyme explorer labeled with third affinity molecules, with the third affinity molecules specifically binding to the first affinity molecules on the other ends of the PCR fragments to form complexes of enzyme explorer-PCR fragment-magnetic bead, and with the complexes of enzyme explorer-PCR fragment-magnetic bead being identified through the enzyme explorer;
wherein the third affinity molecules on the enzyme explorer are different from the second affinity molecules and have specific affinity to the first affinity molecules on the PCR fragments.

2. The method for detecting microorganisms as defined in claim 1, wherein a pair of primer set forth in SEQ ID NO. 1 and 2 is used in the polymerase chain reaction in the step of PCR.

3. The method for detecting microorganisms as defined in claim 2, wherein the pair of primer has the first affinity molecules labeled on 5′ ends.

4. The method for detecting microorganisms as defined in claim 1, wherein the first affinity molecule is biotin.

5. The method for detecting microorganisms as defined in claim 3, wherein the first affinity molecule is biotin.

6. The method for detecting microorganisms as defined in claim 4, wherein the second affinity molecule is streptavidin.

7. The method for detecting microorganisms as defined in claim 1, wherein the third affinity molecule is avidin.

8. The method for detecting microorganisms as defined in claim 1, wherein the enzyme explorer is avidin conjugated horse reddish peroxidase (AV-HRPo).

9. The method for detecting microorganisms as defined in claim 1, wherein the microorganism is hepatitis B virus (HBV).

10. A kit for detecting microorganisms comprising:
a pair of primer, comprising specific sequence to a target microorganism;
a plurality of magnetic beads, having immobilized streptavidin on surfaces thereof; and
an enzyme explorer comprising avidin conjugated luminescences;
wherein the pair of primer has biotin labeled on 5′ ends.

11. The kit for detecting microorganisms as defined in claim 10, wherein the pair of primer is specific to HBV.

12. The kit for detecting microorganisms as defined in claim 11 wherein the pair of primer is set forth in SEQ ID NO. 1 and 2.

13. The kit for detecting microorganisms as defined in claim 10, wherein the enzyme explorer is avidin conjugated horse reddish peroxidase (AV-HRPo).

14. The kit for detecting microorganisms as defined in claim 10, wherein the kit further comprises a washing buffer, a blocking buffer and a binding buffer.
